(19) United States
(12) Patent Application Publication
(10) Pub. No.: US 2015/0072006 A1
(43) Pub. Date: Mar. 12, 2015

(54) Pharmaceutical Formulations

(71) Applicant: Meda Pharma GmbH & Co. KG, Bad Homburg (DE)

(72) Inventors: Katrin Moschner, Dresden (DE); Torsten Hoffmann, Radebeul (DE); Mario Weingart, Dresden (DE); Annegret Hildebrand-Cyrener, Radebeul (DE)

(21) Appl. No.: 14/382,231

(22) PCT Filed: Mar. 1, 2013
(86) PCT No.: PCT/EP2013/000615

§ 371 Date: Aug. 29, 2014

(30) Foreign Application Priority Data
Mar. 2, 2012 (DE) 10 2012 004 065.2

(51) Int. Cl. A61K 9/28; A61K 45/06; A61K 31/44

(52) U.S. Cl. CPC A61K 9/2866; A61K 31/44; A61K 45/06; A61K 9/2846; A61K 9/2853; A61K 9/2813
USPC 424/472; 514/282; 514/295; 514/315; 514/316; 514/326; 514/352; 546/308

(57) ABSTRACT

The invention relates to drug preparations with controlled active ingredient released in the form of microtablets which contain, as active ingredient, flupirtin or one of its physiologically compatible salts, and to processes for their production.

The invention relates to pharmaceutical preparations with controlled-release active ingredient in the form of microtablets comprising flupirtine or a physiologically compatible salt thereof as active ingredient and also methods for the preparation thereof.

Flupirtine (Katadolon™) is a centrally-acting, non-opioid analgesic (Jakoviev, V. Sofia, R. D., Achterrath-Tuckermann, U., von Schlichtegroll, A., Thiemer, K., Arzneim.-Forsch./Drug Res. 35 (I), 30 (1985); Nickel, B., Herz, A., Jakoviev, V., Tibes, U., Arzneim.-Forsch/Drug Res. 35 (I1), 1402 (1985). The centrally-acting analgesic effects of flupirtine operate via other effect mechanisms than the opioid/opiate analgesics (Nickel, B., Postgrad. Med. J. 63 (Suppl. 3), 19 (1987); Szelenyi, I., Nickel, B., Borbe, H. O., Brune K., Br. J. Pharmacol. 143, 89 (1989)). Electrophysiological investigations have shown that flupirtine is able to interfere in the nociceptive response both on the supraspinal and at the spinal level (Carisson, K. H. Jurna, I., Eur. J. Pharmakol. 143, 89 (1987); Bleyer, H., Carlsson K. H., Erkel H. J., Jurna, I., Eur. J. Pharmacol. 151, 259 (1988); Nickel, B., Aledter, A., Postgrad Med. J. 63 (Suppl. 3) 41 (1987)).

Preparations containing flupirtine are part of the prior art. To date flupirtine has been used as therapy in acute pain conditions caused by disease of the musculoskeletal system. In addition, flupirtine has been described in the treatment of tinnitus (WO02/15907), Batten disease (WO01/39760), fibromyalgia (WO00/59487), for cell destruction by apoptosis and necrosis (WO97/49398), impairment of the hemopoietic cell system (WO97/17072), as analgesic (WO97/14415), for neurodegenerative diseases (WO95/05175), for Creutzfeldt-Jacob disease (Molecule of the Month, May 2001) or as anti-inflammatory agent (DE 1795858). Combination drugs of flupirtine with non-steroidal anti-inflammatories are described in EP 189 788. In some indications, it is advantageous that flupirtine has not only an analgesic property but also a muscle-relaxant property, as described in DE-OS 41 22 166.4.

Flupirtine is generally administered orally, rectally or parenterally. Daily doses for oral administration are typically 300-600 mg. To ensure optimal pain treatment, it is desirable to release the drug uniformly over an extended time period in order to reduce the daily doses.

There are no satisfactory drug forms in the prior art which ensure a good yield when manufacturing at low production costs, and offer uniform release, good bioavailability, good dose variability, good processability of the particles by reproducible film-coating and the lowest possible volume of the finished drug form.

Multiparticulate drug forms are often composed of individual particles, granules or pellets, coated with a release-delaying film, which have been compressed into tablets. If these film-coated particles, granules or pellets are compressed to produce a tablet, with or without further auxiliaries, it is possible that the film is damaged by the deformation.

The object of the present invention, therefore, is to provide a multiple unit drug form comprising flupirtine with delayed-release active ingredient having an improved bioavailability and also a more uniform rate of release.

This object is achieved by producing very small tablets, so-called microtablets, on the basis of granules obtained by granulation, preferably dry granulation, which have a high proportion of active ingredient. At least a portion of the microtablets are subsequently coated with a release-delaying film and are optionally filled into a capsule, a sachet, a stick, a pouch or a single-dose dispenser. Due to the high yield, and also a simple production process, they are less expensive to manufacture. Due to the low proportion of auxiliaries, the microtablets have the same amount of active ingredient in a smaller volume and therefore compliance is significantly increased. In addition, their dosage may also be varied and therefore it is possible to manufacture them as a pediatric drug form.

It has been found, surprisingly, that the dry granules may be compressed without difficulty to microtablets, even if the active ingredient content is very high and the conventionally necessary auxiliaries are omitted. Thus, a microtablet preparation is possible with more than 80%, more than 90%, preferably more than 95%, particularly preferably almost 100% of flupirtine maleate. Moreover, the microtablets obtained in this way have a more uniform geometry, more uniform weight and lower porosity (defined surface area) in comparison to granules, active ingredient particles and pellets. A reproducible film-coating process is therefore possible. Granules or also pellets, on the other hand, are often irregular in shape and have a rough and uneven surface, which makes the subsequent film-coating difficult.

In addition, an almost complete processing to microtablets is possible, while for conventional film-coating of pellets and granules a particle size selection (sifting) must be conducted. Otherwise, the variations in the surface to be film-coated are too large and fluctuations in the rate of release occur at the same amount applied. Due to the standardized surface area of the microtablets, the amount of film applied is evenly adjustable which results in a more uniform layer thickness of the film and renders the active ingredient release more reproducible.

If the film-coated microtablets are filled into a capsule, a sachet, a stick, a pouch or a single-dose dispenser, the release-delaying coating is hardly damaged during the dosing process. Thus, a more uniform active ingredient release is ensured and peaks of active ingredient release are avoided (dose dumping).

A major advantage compared to multiple unit tablets is that the release of the active ingredient from the microtablets is independent of the disintegration rate of a tablet in the stomach into the individual delayed-release particles. The influence of food is thus lower, i.e. the rate of passage of the microtablets through the stomach and pylorus to the site of absorption (small intestine) is independent of the state of the stomach content.

The microtablets thus to be produced allow a large variability in the design of the single-dose unit, for example, it is possible to combine microtablets with different active ingredient release profiles (rapid and delayed-release), or even to combine microtablets with various active ingredients which are chemically incompatible with one another, in a capsule, a sachet, a stick, a pouch or a single-dose dispenser.

In addition, the microtablets have the advantage that they are more applicable in cases of difficulties in swallowing than large tablets. The filling of the microtablets into a capsule, a sachet, a stick, a pouch or a single-dose dispenser and the administration with food is unproblematically possible; even administration by gavage tube is possible. The delaying effect is not affected thereby.

The drug form according to the invention is prepared by granulating the active ingredient flupirtine or a pharmaceutically acceptable salt thereof, preferably flupirtine maleate, optionally with addition of magnesium stearate, in a dry granulator (roller compactor). The entire granulate is further processed. The granulate thus obtained is optionally admixed with further auxiliaries, preferably with magnesium stearate, highly-dispersed silicon dioxide, croscarmellose sodium and optionally microcrystalline cellulose and mixed in a container mixer.

This mixture, capable of being tabletted, is compressed on a rotary tablet press to give microtablets having a diameter of 1-5 mm, 1-3 mm, preferably 1.5-2.5 mm, particularly preferably 2 mm.

To prepare the delayed-release microtablets, the microtablets are film-coated in a film-coating apparatus (drum coater, fluidized bed device) with an aqueous suspension of 30% polyacrylate dispersion (Eudragit NM 30 D), talc, yellow iron oxide, polysorbate 80 and hydroxypropylmethyl cellulose. The required active ingredient release profile is achieved by mixing the appropriate proportions of microtablets having delayed-release and immediate-release active ingredient. The single dose of microtablets is filled into a capsule, a sachet, a stick, a pouch or a single-dose dispenser.

The proportion of auxiliaries in the microtablets is between 0.1 and 25% (w/w), preferably between 1 and 20% (w/w), particularly preferably between 3.5 and 5.5% (w/w) or between 11 and 16% (w/w).

The release-delayed coating is between 0.01 and 25% (w/w), between 1 and 15% (w/w), preferably between 4 and 9% (w/w), particularly preferably between 5.5% (w/w) and 7.5% (w/w) of the delayed-release microtablets.

In further embodiments, the delayed-release coating is between 1 and 25% (w/w), between 5 and 10% (w/w), between 10 and 15% (w/w), between 15 and 20% (w/w), between 20 and 25% (w/w), between 5 and 6% (w/w), between 6 and 7% (w/w), between 7 and 8% (w/w), between 8 and 9% (w/w), between 9 and 10% (w/w), between 10 and 11% (w/w), between 11 and 12% (w/w), between 12 and 13% (w/w), between 13 and 14% (w/w), between 14 and 15% (w/w), between 15 and 16% (w/w), between 16 and 17% (w/w), between 17 and 18% (w/w), between 18 and 19% (w/w), between 19 and 20% (w/w), between 20 and 21% (w/w), between 21 and 22% (w/w), between 22 and 23% (w/w), between 23 and 24% (w/w), between 24 and 25% (w/w), particularly preferably at 9.2% (w/w), at 14.2% (w/w), at 19.8% (w/w), at 24.7% (w/w).

By means of the delayed-release coating, a uniform release in vitro of active ingredient from the drug form according to the invention of between 30 and 80% is achieved over 4 hours. According to the composition of the embodiment, i.e. by varying the proportion of flupirtine microtablets with immediate active ingredient release, for which the active ingredient release rate is at least 80% in 45 minutes, release of between 15 and 35% within 30 minutes, or in another embodiment of between 50 and 75% within 4 hours, can be achieved. After a period of 10 hours, the release of the active ingredient is at least 75% of the flupirtine or physiologically compatible salts thereof. Accordingly, a uniform active ingredient level is achieved in vivo. The in vitro release rate of the active ingredient from the pharmaceutical preparation is determined in this case by means of the Ph. Eur. “paddle” method at 100 rpm in a buffer according to Ph. Eur. at a pH of 6.8 at 37° C. by measurement using a UV spectrophotometer.

Further constituents may be all auxiliaries known to those skilled in the art. Swelling agents and binders which may be used are all binders common in pharmaceuticals, preferably cellulose derivatives such as methyl cellulose, hydroxypropyl cellulose, hydroxypropylmethyl cellulose, hydroxyethyl cellulose, carboxymethyl cellulose and salts thereof, alginic acid and salts thereof, propylene glycol alginate, xanthanes, starch, carboxymethyl starch. Different binders, various cellulose derivatives for example, may also be used in parallel at the same time.

Silicon dioxides, silicified microcrystalline cellulose can preferably be used as flow regulators.

Disintegrants preferably used are croscarmellose, starch, cellulose, pectins, alginates, carboxymethyl cellulose, sodium carboxymethyl starch, cross-linked polyvinylpyrrolidone, ultraamylopectin.

Shaping/release agents preferably used are magnesium stearate, stearic acid, talc, calcium stearate, glyceryl behenate, glyceryl monostearate, glyceryl palmitostearate, sodium stearyl fumarate, polyethylene glycols.

Fillers preferably used are starch (e.g. potato starch, corn starch, modified starch), cellulose (e.g. microcrystalline, silicified microcrystalline), calcium hydrogen phosphate and dihydrate thereof, calcium phosphate, lactose, glucose, mannitol, sucrose.

Preferred coating materials for the delayed-release effect are: pure substances or mixtures of hydroxypropylmethyl cellulose phthalate or hydroxypropylmethyl cellulose acetate succinate, cellulose acetate phthalate, starch acetate phthalate and polyvinyl acetate phthalate, carboxymethyl cellulose, polyvinyl acetate, methylcellulose phthalate, methylcellulose succinate, methylcellulose phthalate succinate and methycellulose phthalic acid monoester, zein, ethyl cellulose and ethyl cellulose succinate, shellac, gluten, ethylcarboxyethyl cellulose, ethacrylate-maleic anhydride copolymer, maleic anhydride-vinyl methyl ether copolymer, styrene-maleic acid copolymer, 2-ethylhexyl acrylate-maleic anhydride, crotonic acid-vinyl acetate copolymer, glutamic acid/glutamic ester copolymer, carboxymethylethyl cellulose glycerol monooctanoate, cellulose acetate succinate, polyarginine, fat, oils, waxes, fatty alcohols, anionic polymers of methacrylic acid and methacrylic esters (Eudragit® L, Eudragit® S), copolymers of acrylic and methacrylic esters having a low content of trimethylammonium methacrylate (Eudragit® RL; Eudragt® RS), copolymers of acrylic acid, methacrylic acid and esters thereof (ratio of the free carboxyl groups to the ester groups e.g. 1:1) (Eudragit® L30 D, Eudragit® L100), copolymers of ethyl acrylate and methyl methacrylate (Eudragit® NE 30D, Eudragit NM 30 D), polyvinyl acetate dispersion (Kollicoat® SR 30D), methacrylic acid ethyl acrylate copolymer (Kollicoat® MAE 30 DP, Kollicoat® MAE 100 P).

Preferred plasticizers are, for example, dibutyl sebacate, citric and tartaric esters, glycerol and glycerol esters, phthalic esters.

Furthermore, addition of further functional substances is possible, such as polyethylene glycols, polyvinylpyrrolidone, polyvinyl acetate, polysorbate 80, hydroxypropyl cellulose, hydroxypropylmethyl cellulose, silicon dioxide, dyes.

Preferred release agents and pigments are talc, magnesium stearate, iron oxide, glycerol monostearate, calcium arachinate, glyceryl palmitostearate, stearic acid and triglycerides.

The daily doses are between 20 and 800 mg, between 50 and 600 mg, between 100 and 600 mg, preferably between 300 and 600 mg, particularly preferably 400 mg, or 200-300 mg in children. A single dose is between 20 and 600 mg, preferably 400 mg or 75 mg in children.

The pharmaceutical preparations according to the invention can be used for the treatment of, for example, acute and chronic pain, neuropathic pain, diabetes, ophthalmic diseases, tinnitus, Batten disease, for fibromyalgia, for cell destruction by apoptosis and necrosis, impairment of the hemopoietic cell system, as an analgesic, for neurodegenerative diseases, for Creutzfeldt-Jacob disease, as an anti-inflammatory or as a muscle relaxant.

The pharmaceutical formulations according to the invention may be prepared in a combination with further active ingredients, in particular from the group of the opioids such as, for example, sufentanil, remifentanil, fentanyl, alfentanil, buprenorphine, hydromorphone, levomethadone, oxycodone, diacetylmorphine, methadone, hydrocodone, morphine, piritramide, nalbuphine, pentazocine, codeine, dihydrocodeine, pethidine, tramadol, tilidine, naloxone, naltrexone, loperamide, apomorphine or the pharmaceutically acceptable salts thereof. In one dose unit, any combination of rapid-release and delayed-release microtablets is possible in this context in all active ingredient combinations.

### WORKING EXAMPLES

### Example 1

### Microtablets Comprising Flupirtine Maleate with Immediate-Release Active Ingredient

The active ingredient flupirtine maleate is granulated in a dry granulator (roller compactor). The entire resulting granulate is further processed. 0.012 kg of magnesium stearate, 0.01 kg of highly-dispersed silicon dioxide and 0.02 kg of croscarmellose sodium are added to 0.96 kg of the granulate thus obtained and the mixture is mixed in a container mixer.

This mixture, capable of being tabletted, is compressed on a rotary tablet press to give microtablets having a diameter of 2 mm.

### Example 2

### Microtablets Comprising Flupirtine Maleate with Modified-Release Active Ingredient

600 g of the microtablets, prepared as in example 1, are coated in a film-coating apparatus (drum coater, fluidized bed device) with 58 g of an aqueous suspension of 30% polyacrylate dispersion (Eudragit NM 30 D), 17 g of talc, 1.8 g of yellow iron oxide, 1.7 g of polysorbate 80 and 1.7 g of hydroxypropylmethyl cellulose.

### Example 3

### Microtablets Comprising Flupirtine Maleate with Immediate-Release Active Ingredient

The active ingredient flupirtine maleate is granulated in a dry granulator (roller compactor). The entire resulting granulate is further processed.

0.10 kg of microcrystalline cellulose, 0.012 kg of magnesium stearate, 0.01 kg of highly-dispersed silicon dioxide and 0.01 kg of croscarmellose sodium are added to 0.87 kg of the granulate thus obtained and the mixture is mixed in a container mixer.

This mixture, capable of being tabletted, is compressed on a rotary tablet press to give microtablets having a diameter of 2 mm.

### Example 4

### Microtablets Comprising Flupirtine Maleate with Modified-Release Active Ingredient

600 g of the microtablets, prepared as in example 3, are coated in a film-coating apparatus (drum coater, fluidized bed device) with 58 g of an aqueous suspension of 30% polyacrylate dispersion (Eudragit NM 30 D), 17 g of talc, 1.8 g of yellow iron oxide, 1.7 g of polysorbate 80 and 1.7 g of hydroxypropylmethyl cellulose.

### Example 5

### Microtablets Comprising Tramadol with Immediate-Release Active Ingredient

The active ingredient tramadol hydrochloride is granulated in a dry granulator (roller compactor). The entire resulting granulate is further processed. 0.012 kg of magnesium stearate, 0.01 kg of highly-dispersed silicon dioxide and 0.02 kg of croscarmellose sodium are added to 0.96 kg of the granulate thus obtained and the mixture is mixed in a container mixer.

This mixture, capable of being tabletted, is compressed on a rotary tablet press to give microtablets having a diameter of 2 mm.

### Example 6

### Microtablets Comprising Tramadol with Modified-Release Active Ingredient

600 g of the microtablets, prepared as in example 5, are coated in a film-coating apparatus (drum coater, fluidized bed device) with 58 g of an aqueous suspension of 30% polyacrylate dispersion (Eudragit NM 30 D), 17 g of talc, 1.7 g of polysorbate 80 and 1.7 g of hydroxypropylmethyl cellulose.

### Example 7

### Microtablets Comprising Combination Flupirtine Maleate with Immediate and Modified-Release Active Ingredient

The required active ingredient release profile is achieved by mixing or dosing of the appropriate proportions of microtablets from examples 1 and 2, 1 and 4,1 and 9, 1 and 11, 1 and 13, 1 and 15, 2 and 3, 3 and 4, 3 and 10, 3 and 12, 3 and 14, 3 and 16. The respective single dose of microtablets is filled into a capsule, a sachet, a stick, a pouch or a single-dose dispenser.

### Example 8

### Microtablets Comprising Combination Flupirtine Maleate and Tramadol HCl with Immediate and Modified-Release Active Ingredient

The required active ingredient release profile is achieved by mixing or dosing of the appropriate proportions of microtablets from examples 1 and 5,1 and 6, 2 and 5 or 2 and 6. The respective single dose of microtablets is filled into a capsule, a sachet, a stick, a pouch or a single-dose dispenser.

### Example 9

### Microtablets Comprising Flupirtine Maleate with Modified-Release Active Ingredient

600 g of the microtablets, prepared as in example 1, are coated in a film-coating apparatus (drum coater, fluidized bed device) with 116 g of an aqueous suspension of 30% polyacrylate dispersion (Eudragit NM 30 D), 17 g of talc, 1.8 g of yellow iron oxide, 3.5 g of polysorbate 80 and 3.5 g of silicon dioxide.

### Example 10

### Microtablets Comprising Flupirtine Maleate with Modified-Release Active Ingredient

600 g of the microtablets, prepared as in example 3, are coated in a film-coating apparatus (drum coater, fluidized bed device) with 116 g of an aqueous suspension of 30% polyacrylate dispersion (Eudragit NM 30 D), 17 g of talc, 1.8 g of yellow iron oxide, 3.5 g of polysorbate 80 and 3.5 g of silicon dioxide.

### Example 11

### Microtablets Comprising Flupirtine Maleate with Modified-Release Active Ingredient

600 g of the microtablets, prepared as in example 1, are coated in a film-coating apparatus (drum coater, fluidized bed device) with 232 g of an aqueous suspension of 30% polyacrylate dispersion (Eudragit NM 30 D), 1.8 g of yellow iron oxide, 7 g of polysorbate 80 and 20.9 g of silicon dioxide.

### Example 12

### Microtablets Comprising Flupirtine Maleate with Modified-Release Active Ingredient

600 g of the microtablets, prepared as in example 3, are coated in a film-coating apparatus (drum coater, fluidized bed device) with 232 g of an aqueous suspension of 30% polyacrylate dispersion (Eudragit NM 30 D), 1.8 g of yellow iron oxide, 7 g of polysorbate 80 and 20.9 g of silicon dioxide.

### Example 13

### Microtablets Comprising Flupirtine Maleate with Modified-Release Active Ingredient

600 g of the microtablets, prepared as in example 1, are coated in a film-coating apparatus (drum coater, fluidized bed device) with 348 g of an aqueous suspension of 30% polyacrylate dispersion (Eudragit NM 30 D), 1.8 g of yellow iron oxide, 10.5 g of polysorbate 80 and 31.3 g of silicon dioxide.

### Example 14

### Microtablets Comprising Flupirtine Maleate with Modified-Release Active Ingredient

600 g of the microtablets, prepared as in example 3, are coated in a film-coating apparatus (drum coater, fluidized bed device) with 348 g of an aqueous suspension of 30% polyacrylate dispersion (Eudragit NM 30 D), 1.8 g of yellow iron oxide, 10.5 g of polysorbate 80 and 31.3 g of silicon dioxide.

### Example 15

### Microtablets Comprising Flupirtine Maleate with Modified-Release Active Ingredient

600 g of the microtablets, prepared as in example 1, are coated in a film-coating apparatus (drum coater, fluidized bed device) with 464 g of an aqueous suspension of 30% polyacrylate dispersion (Eudragit NM 30 D), 1.8 g of yellow iron oxide, 14 g of polysorbate 80 and 41.8 g of silicon dioxide.

### Example 16

### Microtablets Comprising Flupirtine Maleate with Modified-Release Active Ingredient

600 g of the microtablets, prepared as in example 3, are coated in a film-coating apparatus (drum coater, fluidized bed device) with 464 g of an aqueous suspension of 30% polyacrylate dispersion (Eudragit NM 30 D), 1.8 g of yellow iron oxide, 14 g of polysorbate 80 and 41.8 g of silicon dioxide.

1. A solid pharmaceutical preparation for oral administration comprising flupirtine or a pharmaceutically acceptable salt thereof, wherein the active ingredient is present in the form of microtablets.

2. The pharmaceutical preparation as claimed in claim 1, wherein at least a portion of the microtablets is present in a delayed-release formulation.

3. The pharmaceutical preparation as claimed in claim 2, wherein the microtablets in the delayed-release formulation are coated with at least one release-delaying component.

4. The pharmaceutical preparation as claimed in claim 3, wherein the coating is present in the form of a film comprising 30% polyacrylate dispersion (Eudragit NM 30 D).

5. The pharmaceutical preparation as claimed in claim 4, wherein the coating additionally comprises one or more auxiliaries from the group comprising talc, iron oxide, polysorbate 80, silicon dioxide and hydroxypropylmethyl cellulose.

6. The pharmaceutical preparation as claimed in claim 3, wherein the release-delaying coating is present between 0.01 and 25% (w/w).

7. The pharmaceutical preparation as claimed in claim 2, wherein a portion of the microtablets is present as an immediate-release formulation.

8. The pharmaceutical preparation as claimed in claim 1, wherein the microtablets have a diameter of 1-5 mm, 1-3 mm, preferably 1.5-2.5 mm, particularly preferably 2 mm.

9. The pharmaceutical preparation as claimed in claim 1, wherein the microtablets are filled in a form suitable for administration as a single dose.

10. The pharmaceutical preparation as claimed in claim 9, wherein the single dose form is a capsule, a sachet, a stick, a pouch or a single-dose dispenser.

11. The pharmaceutical preparation as claimed in claim 1, wherein said preparation comprises at least one further active ingredient.

12. The pharmaceutical preparation as claimed in claim 11, wherein the further active ingredient is selected from the group of the opioids.

13. The pharmaceutical preparation as claimed in claim 12, wherein the further active ingredient is one or more substances selected from the group consisting of sufentanil, remifentanil, fentanyl, alfentanil, buprenorphine, hydromorphone, levomethadone, oxycodone, diacetylmorphine, methadone, hydrocodone, morphine, piritramide, nalbuphine, pentazocine, codeine, dihydrocodeine, pethidine, tramadol, tilidine, naloxone, naltrexone, loperamide, and apomorphine.

14. A method for preparing a pharmaceutical formulation as claimed in claim 1, wherein
(i) the active ingredient is granulated, optionally with addition of suitable auxiliaries
(ii) the granulate is compressed to produce microtablets, optionally with addition of suitable auxiliaries
(iii) the microtablets obtained are optionally evenly coated with a release-delaying layer.

15. A method of treating a condition comprising administering the pharmaceutical preparation as claimed in claim 1, wherein the condition is selected from the group consisting of acute and chronic pain, neuropathic pain, diabetes, ophthalmic diseases, tinnitus, Batten disease, fibromyalgia, diseases associated with cell destruction by apoptosis and necrosis, diseases with impairment of the hemopoietic cell system, neurodegenerative diseases, Creutzfeldt-Jacob disease, inflammatory disorders and muscle tension.
